(19) United States
(12) Patent Application Publication
(10) Pub. No.: US 2026/0345241 A1
(43) Pub. Date: Nov. 5, 2026

(54) METHODS FOR PREDICTING PROSTHETIC VALVE OUTFLOW TRACT OBSTRUCTION

(71) Applicant: Medtronic, Inc., Minneapolis, MN (US)

(72) Inventors: Priya NAIR, Santa Rosa, CA (US); Madeline M. TUNENDER, Santa Ana, CA (US); Sarah E. AHLBERG, Mounds View, MN (US)

(21) Appl. No.: 19/100,189

(22) PCT Filed: Jul. 31, 2023
(86) PCT No.: PCT/US2023/071345

§ 371 Date: Jan. 31, 2025

Related U.S. Application Data
(60) Provisional application No. 63/394,129, filed on Aug. 1, 2022.

(51) Int. Cl. A61B 34/10

(52) U.S. Cl. CPC A61B 34/10; A61B 2034/102; A61B 2034/104; A61B 2034/108

(57) ABSTRACT

Systems and methods for evaluating a subject having indications for receiving a prosthetic valve, such as a prosthetic mitral valve. Images of the subject's native annulus are obtained and assessed. A candidate prosthetic valve is selected based upon the assessment. An implant model of the candidate prosthetic valve is selected from a library of different implant models based upon the native annulus assessment. A virtual implant representation is generated by applying the selected implant model to the obtained images. An area of the neo-VOT of the virtual implant representation is reviewed. Whether or not the candidate prosthetic valve is appropriate for the subject is evaluated based upon the review.

### FIELD

The present disclosure relates to systems and methods for evaluating or predicting potential obstructions to blood through an anatomical structure of interest. More particularly, it relates to systems and methods for predicting or evaluating the potential effects of the placement of a prosthetic device, for example possible left ventricular outflow tract area reductions potentially created by placement of a candidate prosthetic mitral valve.

### BACKGROUND

A human heart includes four heart valves that determine the pathway of blood flow through the heart: the mitral valve, the tricuspid valve, the aortic valve, and the pulmonary valve. The mitral and tricuspid valves are atrio-ventricular valves, which are between the atria and the ventricles, while the aortic and pulmonary valves are semilunar valves, which are in the arteries leaving the heart. Ideally, native leaflets of a heart valve move apart from each other when the valve is in an open position, and meet or “coapt” when the valve is in a closed position. Problems that may develop with valves include stenosis in which a valve does not open properly, and/or insufficiency or regurgitation in which a valve does not close properly. Stenosis and insufficiency may occur concomitantly in the same valve. The effects of valvular dysfunction vary, with regurgitation or backflow typically having relatively severe physiological consequences to the patient.

Diseased or otherwise deficient heart valves can be repaired or replaced using a variety of different types of heart valve surgeries. One conventional technique involves an open-heart surgical approach that is conducted under general anesthesia, during which the heart is stopped and blood flow is controlled by a heart-lung bypass machine.

More recently, minimally invasive approaches have been developed to facilitate catheter-based implantation of the valve prosthesis on the beating heart, intending to obviate the need for the use of classical sternotomy and cardiopulmonary bypass. In general terms, an expandable prosthetic valve is compressed about or within a catheter, inserted inside a body lumen of the patient, such as the femoral artery, and delivered to a desired location in the heart.

The heart valve prosthesis employed with catheter-based, or transcatheter, procedures generally includes an expandable multi-level frame or stent that supports a valve structure having a plurality of leaflets. The frame can be contracted during transluminal delivery, and expanded upon deployment at or within the native valve. One type of valve stent can be initially provided in an expanded or uncrimped condition, then crimped or compressed about a balloon portion of a catheter. The balloon is subsequently inflated to expand and deploy the prosthetic heart valve. With other stented prosthetic heart valve designs, the stent frame is formed to be self-expanding. With these systems, the valved stent is crimped down to a desired size and held in that compressed state within a sheath or capsule for transluminal delivery. Retracting or extending the sheath or capsule from this valved stent allows the stent to self-expand to a larger diameter, fixating at the native valve site. In more general terms, then, once the prosthetic valve is positioned at the treatment site, for instance within an incompetent native valve, the stent frame structure may be expanded to hold the prosthetic valve firmly in place. One example of a stented prosthetic valve is disclosed in U.S. Pat. No. 5,957,949 to Leonhardt et al., which is incorporated by reference herein in its entirety.

Patient screening for a prosthesis, such as a prosthetic heart valve, can be challenging due to the anatomical complexities of the patient population. Some screening processes may be costly, time-consuming, subjective, and not sufficiently predictive. For example, some screening processes for predicting left ventricular outflow tract (LVOT) areas associated with a candidate prosthetic mitral valve may incorrectly predict LVOT obstruction. Similar concerns may arise with respect to predicting right ventricular outflow tract (RVOT) areas associated with a candidate prosthetic tricuspid valve.

The present disclosure addresses problems and limitations associated with the related art.

### SUMMARY

Some aspects of the present disclosure are directed to methods for evaluating a human subject having indications for receiving a prosthetic valve. The methods include obtaining images of a native valve and surrounding anatomy of the subject. A native annulus of the native valve in the obtained images is assessed. For example, dimensions or geometric attributes mitral annulus can be determined, such as anterior-posterior diameter, commissure-commissure diameter, perimeter, etc. A candidate prosthetic valve is selected from a plurality of available prosthetic valves based upon the native annulus assessment. An implant model of the candidate prosthetic valve is selected from a library of different implant models based upon the native annulus assessment. A virtual implant representation is generated by applying or overlaying the selected implant model to the obtained images, the virtual implant representation having a neo-VOT (ventricular outflow tract). An area of the neo-VOT is reviewed. Whether or not the candidate prosthetic valve is appropriate for the subject is evaluated based upon the review. For example, where a minimum area of the neo-VOT is determined to be less than a threshold or cutoff criteria, it can be determined that the candidate prosthetic valve is not appropriate for the subject. In some embodiments, the methods of the present disclosure can be useful in screening potential prosthetic mitral valve recipients in which a neo-LVOT (left ventricular outflow tract) is reviewed and/or in screening potential tricuspid valve recipients in which a neo-RVOT (right ventricular outflow tract) is reviewed.

### BRIEF DESCRIPTION OF DRAWINGS

FIG. 1 is a schematic sectional illustration of a mammalian heart having native valve structures;

FIG. 2A is a schematic sectional illustration of a left ventricle of a mammalian heart showing anatomical structures and a native mitral valve;

FIG. 2B is a schematic sectional illustration of the left ventricle of the heart having a prolapsed mitral valve in which the leaflets do not sufficiently coapt and which may be suitable for replacement with a prosthetic heart valves;

FIG. 3A is a schematic illustration of a superior view of a mitral valve isolated from the surrounding heart structures and showing the annulus and native leaflets;

FIG. 3B is a schematic illustration of a superior view of a mitral valve, aortic mitral curtain and portions of the aortic valve isolated from the surrounding heart structures and showing regions of the native mitral valve leaflets;

FIG. 4A is an image of a section of a human heart and highlighting a native left ventricular outflow tract (LVOT);

FIG. 4B is an image of a section of the human heart of FIG. 4A and highlighting a neo-LVOT created by a prosthetic mitral valve frame;

FIG. 5 is a block diagram illustrating a computing system for evaluating a human subject having indications for receiving a prosthetic mitral valve;

FIG. 6 is a flow diagram illustrating a method of evaluating a human subject having indications for receiving a prosthetic mitral valve;

FIG. 7 is a flow diagram illustrating additional details of the method of FIG. 6;

FIGS. 8A-8C are images illustrating techniques for assessing a mitral annulus of a subject and useful with the methods of FIG. 6;

FIG. 9 is a lookup table useful for identifying an implant model from a library of implant models and useful with the methods of FIG. 6;

FIGS. 10A-10E illustrate a method for generating an implant model in accordance with some embodiments of the present disclosure;

FIG. 11 is an example virtual implant representation provided by systems and methods of the present disclosure; and

FIGS. 12A-12D are images illustrating methods for reviewing a neo-LVOT of a virtual implant representation in accordance with principles of the present disclosure.

### DETAILED DESCRIPTION

Specific embodiments of the present disclosure are now described with reference to the figures, wherein like reference numbers indicate identical or functionally similar elements.

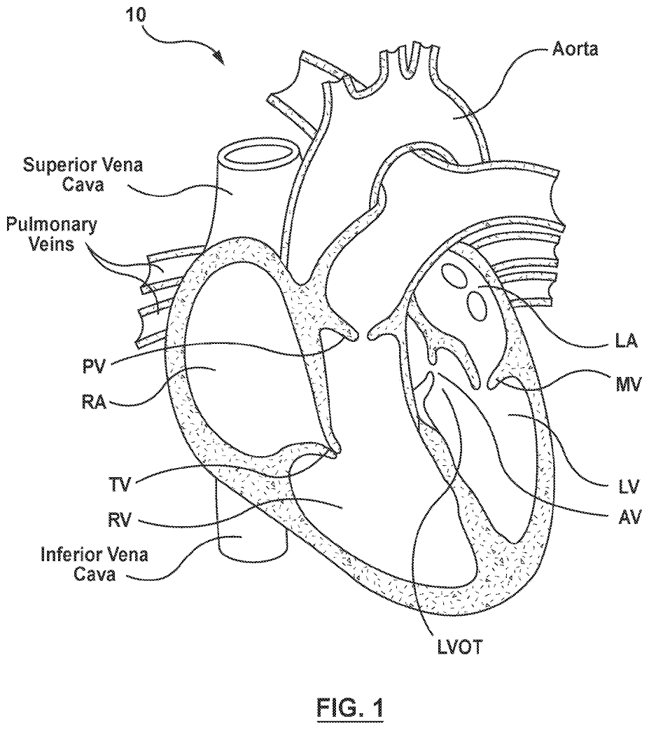

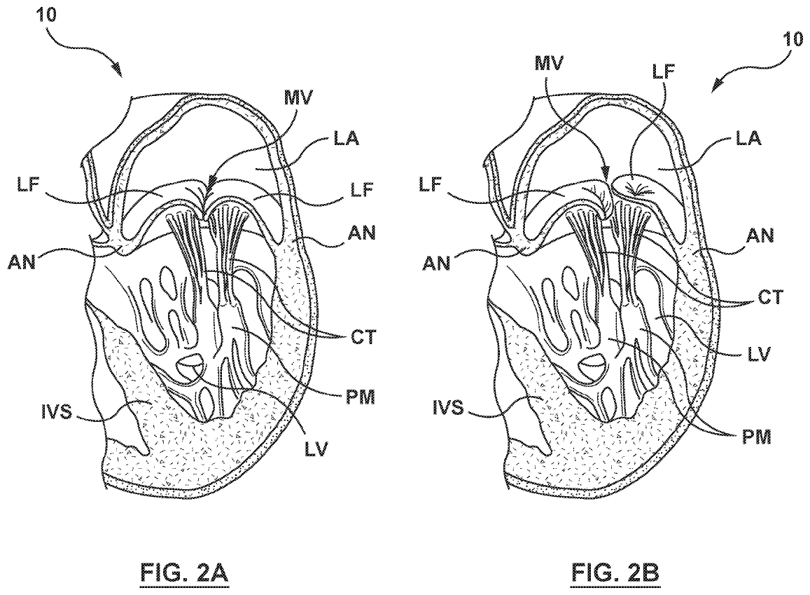

FIG. 1 is a schematic sectional illustration of a mammalian heart 10 that depicts the four heart chambers (right atria RA, right ventricle RV, left atria LA, left ventricle LV) and native valve structures (tricuspid valve TV, mitral valve MV, pulmonary valve PV, aortic valve AV). FIG. 2A is a schematic sectional illustration of a left ventricle LV of a mammalian heart 10 showing anatomical structures and a native mitral valve MV. Referring to FIGS. 1 and 2A together, the heart 10 comprises the left atrium LA that receives oxygenated blood from the lungs via the pulmonary veins. The left atrium LA pumps the oxygenated blood through the mitral valve MV and into the left ventricle LV during ventricular diastole. The left ventricle LV contracts during systole and blood flows outwardly through the aortic valve AV, into the aorta and to the remainder of the body.

In a healthy heart, the leaflets LF of the mitral valve MV meet evenly at the free edges or “coapt” to close and prevent back flow of blood during contraction of the left ventricle LV (FIG. 2A). Referring to FIG. 2A, the leaflets LF attach the surrounding heart structure via a dense fibrous ring of connective tissue called an annulus AN which is distinct from both the leaflet tissue LF as well as the adjoining muscular tissue of the heart wall. In general, the connective tissue at the annulus AN is more fibrous, tougher and stronger than leaflet tissue. The flexible leaflet tissue of the mitral leaflets LF are connected to papillary muscles PM, which extend upwardly from the lower wall of the left ventricle LV and the interventricular septum IVS, via branching tendons called chordae tendinae CT. In a heart 10 having a prolapsed mitral valve MV in which the leaflets LF do not sufficiently coapt or meet, as shown in FIG. 2B, leakage from the left atrium LA into the left ventricle LV will occur. Several structural defects can cause the mitral leaflets LF to prolapse such that regurgitation occurs, including ruptured chordae tendinae CT, impairment of papillary muscles PM (e.g., due to ischemic heart disease), and enlargement of the heart and/or mitral valve annulus AN (e.g., cardiomyopathy).

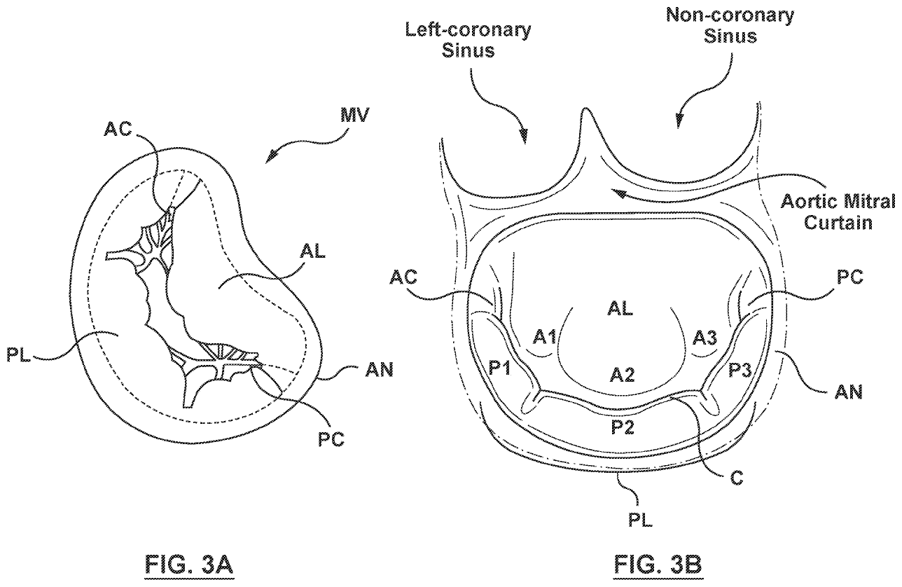

FIG. 3A is a superior view of a mitral valve MV isolated from the surrounding heart structures and further illustrating the shape and relative sizes of the mitral valve leaflets AL, PL and annulus AN. FIG. 3B is a schematic illustration of a superior view of a mitral valve MV, aortic mitral curtain and portions of the aortic valve AV isolated from the surrounding heart structures and showing regions of the native mitral valve leaflets AL, PL. With reference to FIGS. 3A and 3B together, the mitral valve MV includes an anterior leaflet AL with segments or scallops A1, A2, and A3 that meet and oppose respective segments or scallops P1, P2 and P3 of a posterior leaflet PL at a coaptation line C (FIG. 3B) when closed. FIGS. 3A and 3B together further illustrate the shape and relative sizes of the leaflets AL, PL of the mitral valve. As shown, the mitral valve MV generally has a “D” or kidney-like shape and the line of coaptation C is curved or C-shaped, thereby defining a relatively large anterior leaflet AL and substantially smaller posterior leaflet PL. Both leaflets appear generally crescent-shaped from the superior or atrial side, with the anterior leaflet AL being substantially wider in the middle of the valve at the A2 segment thereof than the posterior leaflet at the P2 segment thereof (e.g., comparing segments A2 and P2, FIG. 3B). As illustrated in FIGS. 3A and 3B, at the opposing ends of the line of coaptation C, the leaflets join together at corners called the anterolateral commissure AC and posteromedial commissure PC, respectively. When the anterior leaflet AL and posterior leaflet PL fail to meet (FIG. 3A), regurgitation between the leaflets AL, PL or at commissures AC, PC at the corners between the leaflets can occur.

Referring to FIGS. 3A and 3B, the mitral valve annulus AN is a fibrotic ring that consists of an anterior part and a posterior part. The aortic-mitral curtain (FIG. 3B) is a fibrous structure that connects the anterior mitral annulus AN intimately with the aortic valve annulus (at the level of the left and non-coronary cusps or sinuses). The posterior part of the mitral annulus AN is not reinforced by other structures of the heart and is rather discontinuous (making it prone to dilatation). The leaflets AL, PL and the annulus AN are comprised of different types of cardiac tissue having varying strength, toughness, fibrosity, and flexibility. Furthermore, the mitral valve MV may also comprise a region of tissue interconnecting each leaflet to the annulus AN (indicated at dashed line in FIG. 3A).

A person of ordinary skill in the art will recognize that the dimensions and physiology of the patient may vary among patients, and although some patients may comprise differing physiology, the teachings as described herein can be adapted for use by many patients having various conditions, dimensions and shapes of the mitral valve. For example, work in relation to embodiments suggests that patients may have a long dimension across the annulus and a short dimension across the annulus with or without well-defined peak and valley portions

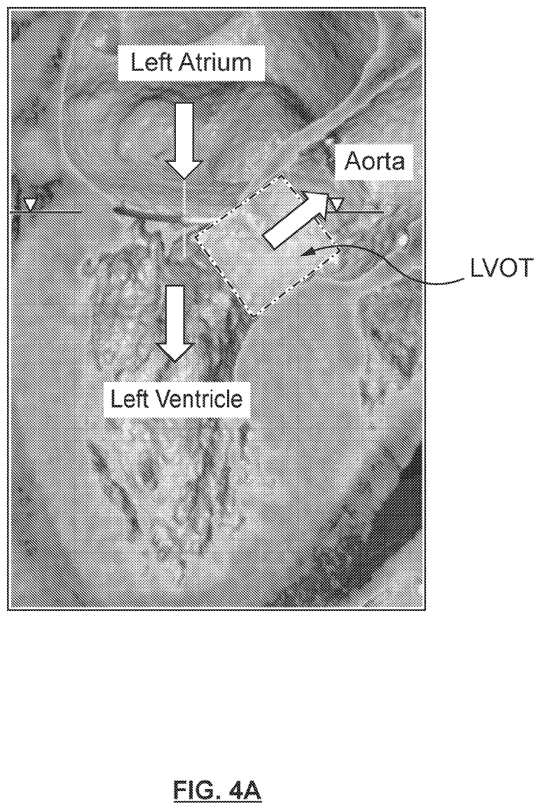

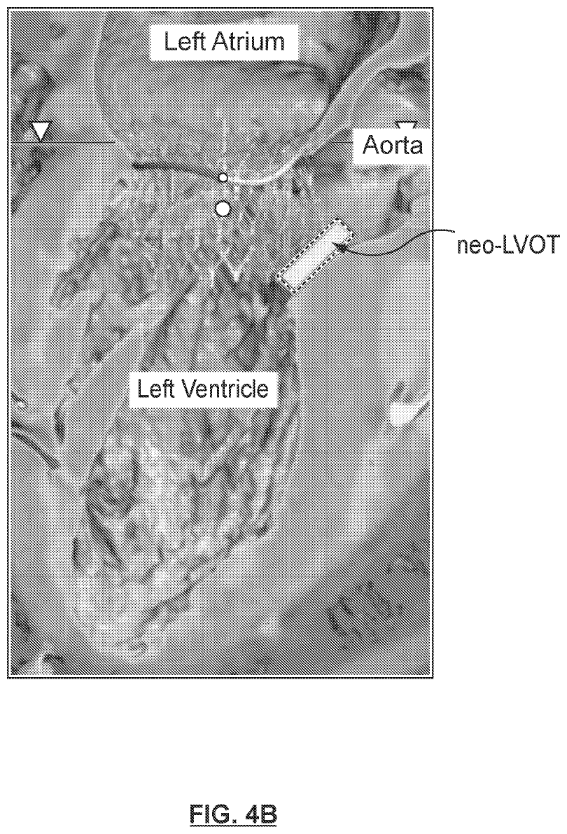

The left ventricular outflow tract LVOT is generally identified in FIG. 1 and is that portion of the left ventricle LV where blood flows out of the heart and into the aorta. As highlighted in the image of FIG. 4A, the native LVOT is understood to be the region between the basal septum of the left ventricle LV and the anterior leaflet of the mitral valve MV.

Any obstruction to the LVOT can compromise the amount of blood flowing out of the heart. LVOT obstruction is a potential complication resulting from transcatheter mitral valve replacement, mitral valve-in-valve, and valve-in-ring procedures, as well as implantation of transcatheter heart valves in calcific mitral valve disease. In some instances, an implanted transcatheter mitral valve replacement device can project or protrude into the left ventricle in a manner that modifies the native LVOT, creating a new or neo-LVOT as highlighted, for example, in the image of FIG. 4B. The implanted device can sometime cause LVOT obstruction (LVOTO) in certain anatomies.

Clinically, LVOTO is identified by the presence of high gradients across the LVOT; however, there is a lack of consensus on the exact grading criteria. The Mitral Valve Academic Research Consortium defines LVOTO as an increase in gradient≥10 mm Hg from baseline, while other papers have defined LVOTO as peak gradients≥30 mm Hg or 50 mm Hg. In the context of transcatheter mitral valve replacement, there are a number of factors that contribute to LVOTO, including anatomical risk factors, device-related risk factors, procedural risk factors, and clinical risk factors. Clinicians often seek to account for anatomical and device-related risk when assessing a prospective transcatheter mitral valve replacement patient. For example, as part of a patient screening process, clinicians can seek to predict or estimate the neo-LVOT in a prospective patient were a transcatheter mitral valve replacement device implanted, and further evaluate the so-predicted neo-LVOT for clinically relevant obstruction or LVOTO. Where the predicted neo-LVOT is determined to implicate an LVOTO concern, the prospective patient can be characterized as having failed the LVOTO patient screening and excluded from further consideration for transcatheter mitral valve replacement.

Against the above background, some embodiments of the present disclosure relate to systems (e.g., computing systems) and methods for evaluating a human subject having indications for receiving a prosthetic valve, such as a prosthetic mitral valve (e.g., a transcatheter mitral valve replacement (TMVR) device) or a prosthetic tricuspid valve. In general terms, some methods of the present disclosure entail generating a virtual implant representation by applying an implant model of a prosthetic mitral valve to images of the subject's native mitral valve and surrounding anatomy. The virtual implant representation reflects a neo-LVOT that can be reviewed. From this review, an evaluation is made as to whether or not the prosthetic mitral valve is appropriate for the subject, for example if the evaluation reveals a possible LVOTO concern. As described in greater detail below, the implant model is representative of an actual or available prosthetic mitral valve and is selected from a library of models based, at least in part, upon an assessment of the subject's native mitral valve annulus. With the systems and methods of the present disclosure, highly viable predictions of a subject's post-implant neo-LVOT are provided and from which a reliable LVOTO evaluation can be made. In other embodiments, similar systems and methods can be used to provide a virtual implant representation of a prosthetic tricuspid valve and from which a neo-RVOT can be reviewed for possible RVOT obstruction concerns.

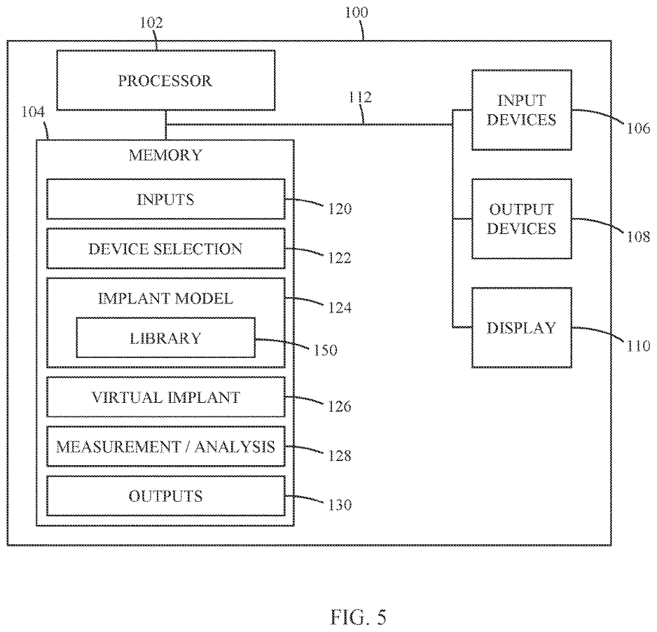

FIG. 5 is a block diagram illustrating a computing system 100 for evaluating a human subject (or patient) having indications for receiving a prosthetic mitral valve (e.g., a transcatheter mitral valve replacement, or TMVR, device) according to one embodiment. The system 100 includes a processor 102, a memory 104, input devices 106, output devices 108, and a display 110. The processor 102, memory 104, input devices 106, output devices 108, and display 110 are communicatively coupled to each through a communication link 112.

The input devices 106 can include one or more of a keyboard, mouse, data ports, stylus and/or other suitable devices for inputting information into the system 100. The output devices 108 can include one or more of speakers, data ports, and/or outer suitable devices for outputting information from the system 100. The display 110 can be any type of display device that displays information to a user of the system 100.

The processor 102 includes a central processing unit (CPU) or other suitable processor. In an example, the memory 104 stores machine readable instructions executed by the processor 102 for operating the system 100. The memory 104 includes any suitable combination of volatile and/or non-volatile memory, such as combinations of random-access memory (RAM), read-only memory (ROM), flash memory, and/or other suitable memory. These are examples of non-transitory computer readable media (e.g., non-transitory computer-readable storage media storing computer-executable instructions that when executed by at least one processor cause the at least one processor to perform a method). The memory 104 is non-transitory in the sense that it does not encompass a transitory signal but instead is made up of at least one memory component to store machine executable instructions for performing techniques or methodologies described herein.

The memory 104 stores inputs 120, a device selection module 122, an implant model library module 124, a virtual implant module 126, a measurement and analysis module 128, and outputs 130. Processor 102 executes instructions of modules 122, 124, 126, 128 to perform techniques described herein based on the inputs 120 to generate the outputs 130. In some embodiments, the inputs 120 include obtained images of a native mitral valve and surrounding anatomy of a subject. The device selection module 122 selects, or facilitates user selection of, a candidate prosthetic mitral valve from a plurality of differently sized and/or shaped prosthetic mitral valves. The implant model library module 124 selects, or facilitates user selection of, an implant model of the candidate prosthetic mitral valve. The virtual implant module 126 generates a simulated positioning of the candidate prosthetic mitral valve to the subject's native mitral valve anatomy images. The measurement and analysis module 128 measures or estimates a neo-LVOT associated with the simulated positioning of the candidate mitral valve. Results from one or more of the modules 122-128 can be provided to a user as the outputs 130

In some examples, the various subcomponents or elements of the system 100 may be embodied in a plurality of different systems, whereas modules may be grouped or distributed across the plurality of different systems. To achieve its desired functionality, the system 100 may include various hardware components. Among these hardware components may be a number of processing devices, a number of data storage devices, a number of peripheral device adaptors, and a number of network adaptors. These hardware components may be interconnected through the use of busses and/or network connections. The processing devices may include a hardware architecture to retrieve executable code from the data storage devices and execute the executable code. The executable code may, when executed by the processing devices, cause the processing devices to execute some of the functionality disclosed herein.

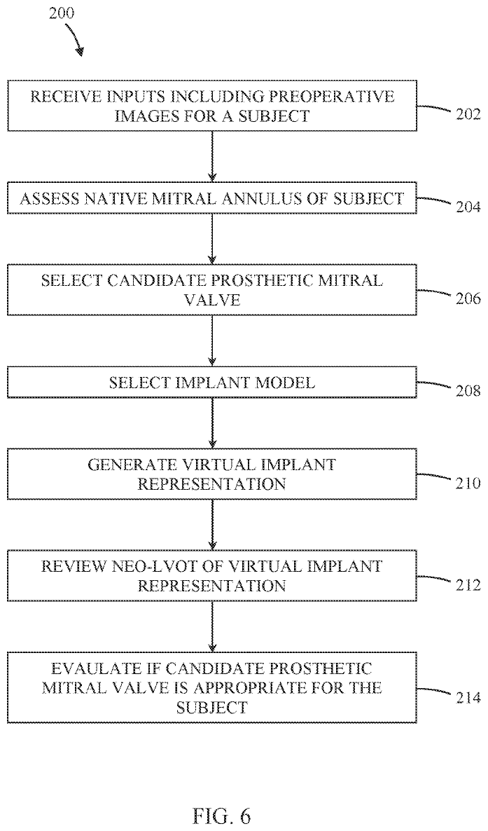

FIG. 6 is a flow diagram illustrating a method 200 according to certain embodiments. In some embodiments, computing system 100 (FIG. 5) is configured to perform one or more or all steps of the method 200. It should be noted that in certain embodiments, method 200 is a computer-implemented method or process. Further, certain blocks may be performed automatically, manually by user of a computing device, or partially manually and partially automatically such as based on input from a user of a computing device. Further, certain blocks may be optional, and parts of the described method may be performed as separate methods. At a step 202, the method 200 includes receiving preoperative images for a human subject. The images include or relate to a native mitral valve and surrounding anatomy of the subject. At a step 204, the method includes assessing a native annulus of the native mitral valve based on the obtained images. At a step 206, a candidate prosthetic mitral valve is selected from a plurality of available prosthetic mitral valves based upon the native annulus assessment. The assessment at the step 204 and/or the selection at the step 206 may be performed by the device selection module 122 (FIG. 5). At a step 208, an implant model of the selected candidate prosthetic mitral valve is selected from a library of different implant models based upon the native annulus assessment. The selection at the step 208 may be performed by the implant model library module 124 (FIG. 5). At a step 210, a virtual implant representation is generated, for example by applying or overlaying the selected implant model to the obtained images. The virtual implant representation implicates a likely arrangement of the selected candidate prosthetic mitral valve to the subject's native were the candidate prosthetic mitral valve actually implanted, and has an identifiable neo-LVOT. The virtual implant representation at the step 210 can be performed by the virtual implant module 126 (FIG. 5). At 212, an area of the neo-LVOT of the virtual implant representation is reviewed. Based upon this review, whether or not the candidate prosthetic mitral valve is appropriate for the subject is determined at 214. One or both of the review at 212 and the evaluation at 214 may be performed by the measurement and analysis module 128 (FIG. 5).

The native mitral valve and surrounding anatomy images of the subject provided at step 202 can be obtained in various manners. In some embodiments, data representative of a subject-specific, three-dimensional (3D) image of a cardiac region including the native mitral valve is provided to the processor 102, for example obtained by computer tomography (CT) or magnetic resonance imaging (MRI). Thus, the data can be one or more 3D CT images and/or one or more 3D MRI images of the cardiac region of the subject. Thus, in some embodiments, the inputs 120 can include a medical image device and/or a database of obtained medical images (e.g., single phase CT images or multiphase CT images imported to the system 100).

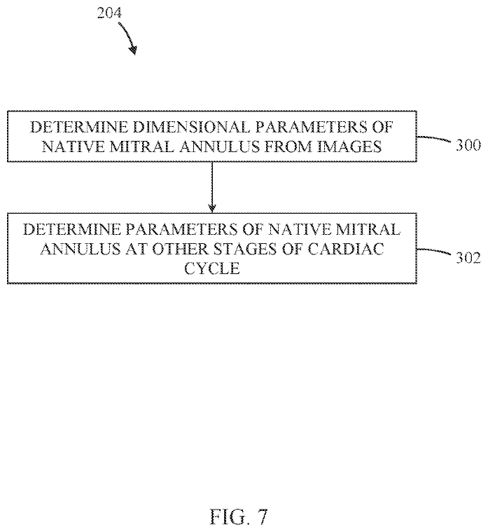

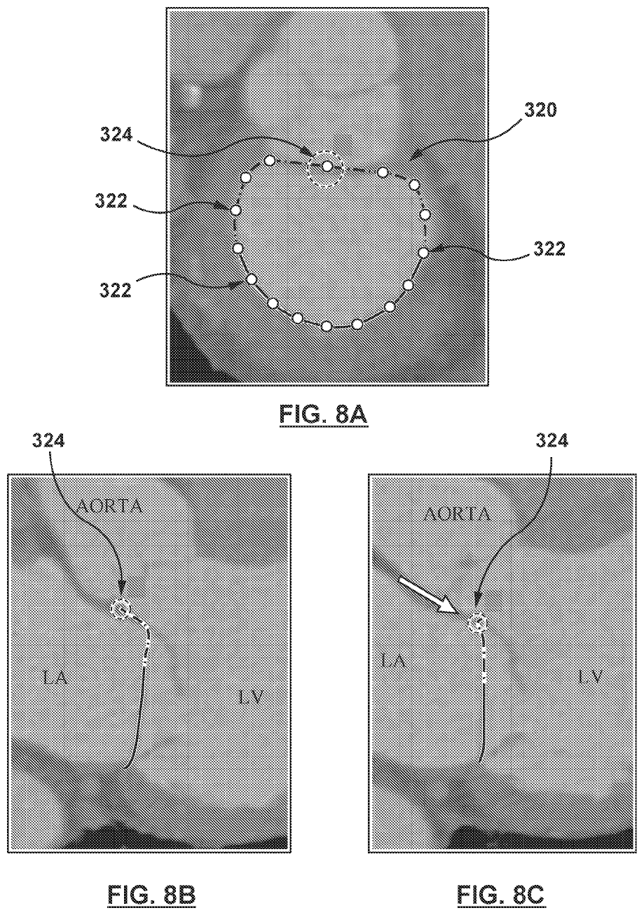

The step 204 of assessing the annulus of the native mitral valve in the obtained image(s) can include or incorporate various techniques or processes that generate information useful for selecting an appropriately-sized, candidate prosthetic mitral valve. In some embodiments, the step 204 of assessing can include evaluating or determining dimensions of the native mitral annulus. For example, and with reference to FIG. 7, at 300 one or more dimensional parameters of the native mitral annulus are determined. The dimensional parameters can include annulus perimeter, anterior-posterior diameter, commissure-commissure diameter, and/or annulus area. The dimensional parameter(s) can be determined in various fashions. By way of non-limiting example, FIG. 8A is a CT image of a subject's cardiac anatomy that includes a native mitral annulus 320. The native mitral annulus 320 has been defined in the image via the overlaying or placement of points 322 (several of which are labeled in FIG. 8A) along or around the native mitral annulus 320. In some examples, sixteen of the points 322 can be used, although any other number, either greater or lesser, is also acceptable. To facilitate a more useful assessment of the native mitral annulus 320, an anterior (generally labeled at 324 in FIG. 8A) of the native mitral annulus 320 is truncated at the trigones, as generally reflected by FIGS. 8B and 8C. As a point of reference, truncation can affect the position of the mitral valve plane, thereby affecting the location of prosthetic mitral valve placement. Regardless, and returning to FIG. 8A, the dimensions or parameters of interest can be derived from the mitral valve annulus as defined by the points 322.

It will be understood that the image of FIG. 8A represents a single stage of a complete cardiac cycle. With this in mind, and as indicated at 302 in FIG. 7, the one or more dimensional parameters of the native annulus are further determined at other stages of the cardiac cycle, for example by repeating the evaluation or measuring processes described above with respect images of the subject's native mitral annulus at other cardiac stages. In some examples, the evaluations or measurements are generated for all ten phases of the cardiac cycle, in 10% increments.

Returning to FIG. 6, the step 206 of selecting a candidate prosthetic mitral valve from a plurality of available prosthetic mitral valves based upon the native annulus assessment can be performed in various manners. As a point of reference, a clinician or clinical resource involved in the evaluation of a subject for potentially receiving a prosthetic mitral valve will typically have a number of similarly designed (or styled), but differently-sized prosthetic mitral valves available for use with various patients. By way of non-limiting example, two differently-sized prosthetic mitral valves (or transcatheter mitral valve replacement systems) are available under the tradename Intrepid™ from Medtronic, Inc., with a diameter on the order of 42 millimeters (mm) or 48 mm. A greater number of prosthetic mitral valves may be available in other embodiments that may or may not have the same design/style. A number of other or different post-crimp diameter prosthetic mitral valves may be available in other embodiments (in addition or as an alternative to the 42 mm and 48 mm examples).

Various criteria can be employed in selecting a candidate prosthetic mitral valve from the plurality of available prosthetic mitral valves. In some embodiments, the device selection module 122 (FIG. 5) has access to or maintains a database or library of relevant parameter(s) (e.g., dimensions) of the available prosthetic mitral valves, and is programmed to, or operates one or more protocols or algorithms formatted to, apply determined criteria. In some examples, the selection criteria can be based on perimeter oversizing. For example, a primary criteria for selecting a candidate prosthetic mitral valve is that a perimeter of the candidate prosthetic mitral valve is 10%-30% greater than a perimeter of the native mitral annulus (as determined above). Average anterior-posterior (AP) diameter, commissure-commissure (CC) diameter and/or equivalent diameter (i.e., (AP+CC)/2) can also be determined and reviewed for oversizing criteria. In some examples, Equation 1 (below) can be used to determine oversizing:

$$oversize = \frac{( prosthesis dimension - a v erage native annulus dimension )}{average native annulus dimension} \times 100$$

Regardless of how the candidate prosthetic mitral valve is chosen, the subsequent step 208 of selecting an implant model of the candidate prosthetic mitral valve can be performed in various manners. In some embodiments, the implant model library module 124 can have access to or maintain a library 150 of different virtual implant models that otherwise represent an expected geometry or footprint of a corresponding prosthetic mitral valve upon final implant. The step 208 can include selecting the virtual implant from the library 150 that most closely corresponds to the candidate prosthetic mitral valve and dimensional parameter(s) of the subject's native mitral annulus. In some embodiments, various ones of the virtual implant models in the virtual implant model library 150 relate to a corresponding one of the available prosthetic mitral valves of the available prosthetic mitral valves discussed above. By way of example, where the plurality of available prosthetic mitral valves includes a prosthetic mitral valve A (“PMV-A”) and a prosthetic mitral valve B (“PMV-B”), and at least one feature or parameter of PMV-A differs from that of PMV-B (e.g., different diameters), the virtual implant model library 150 includes at least one virtual implant model (“Model-A”) of PMV-A and at least one virtual implant model (“Model-B”) of PMV-B.

In some embodiments, the virtual implant model library 150 includes two or more different virtual representations or models of at least one of the available prosthetic mitral valves, with each virtual model representing the available prosthetic mitral valve upon final deployment to a differently-configured native mitral annulus. By way of reference, geometries or three-dimensional attributes of a mitral annulus can substantively vary from person-to-person. Thus, the footprint or spatial arrangement of a prosthetic mitral valve upon final deployment can vary from one particular mitral valve to another. With this in mind, in some embodiments, the virtual implant model library 150 can include virtual representations of an available prosthetic mitral valve upon final deployment within a plurality of different mitral annulus-like shaped openings each with one or more defined or known geometries or geometric attributes. Thus, and continuing the example above, the virtual implant model library 150 can include first, second, . . . n virtual implant models (Model-A1, Model-A2, . . . . Model-An) of PMV-A, where Model-A1 represents PMV-A deployed within a first mitral annulus-like shaped opening having one or more known geometric attributes, Model-A2 represents PMV-A deployed within a second mitral annulus-like shaped opening having one or more known geometric attributes differing from those of the first opening, etc. Where the candidate prosthetic mitral valve selected at the step 206 is PMV-A, the step 208 can include comparing assessed geometric attributes of the subject's native mitral annulus with the known geometric attribute(s) of Model-A1 . . . . Model-An. The virtual implant model Model-A1 . . . . Model-An most closely resembling the assessed geometric attributes of the subject's native mitral annulus can then be identified as the selected virtual model for subsequent processes described below.

The library 150 can similarly include a plurality of different virtual representations for one or more additional available prosthetic mitral valves that are available for selection. From the example above, the library 150 can include first, second, . . . n virtual implant models (Model-B1, Model-B2, . . . . Model-Bn) of PMV-B, where Model-B1 represents PMV-B deployed within a first mitral annulus-like shaped opening having one or more known geometric attributes, Model-B2 represents PMV-B deployed within a second mitral annulus-like shaped opening having one or more known geometric attributes differing from those of the first opening, etc. Where the candidate prosthetic mitral valve selected at the step 206 is instead PMV-B, the step 208 can entail comparing assessed geometric attributes of the subject's native mitral annulus with the known geometric attribute(s) of Model-B1 . . . . Model-Bn, and then selecting the “closest” or “best fit” virtual implant model.

Various known geometric attributes can be embodied by the virtual implant models and serve as a basis for model selection. In some embodiments, the virtual implant models can each represent the corresponding available prosthetic mitral valve deployed within a mitral annulus-like shaped opening having a known ellipticity. In some embodiments, “ellipticity” is the major diameter of the opening divided by the minor diameter of the opening, and is sometimes referred to as an “ellipticity index”.

An additional known geometric attribute that can be embodied by each of the virtual implant models is “oversizing” or an “oversizing value”. As mentioned above, “oversizing” is in reference to the prosthetic mitral valve being slightly larger than the native mitral annulus to which the prosthetic mitral valve is deployed. For example, “oversizing” can be in reference to dimensions of the prosthetic mitral valve at an engagement region thereof (i.e., that region of the prosthetic mitral valve intended to be in contact with the native mitral annulus upon final implant) being slightly greater than one or more of the diameter(s) (e.g., anterior-posterior diameter, commissure-commissure diameter), perimeter, area, etc., of the native mitral annulus. In some embodiments, then, the virtual implant models can each represent the corresponding available prosthetic mitral valve deployed within a mitral annulus-like shaped opening, where dimension(s) of the engagement region of the prosthetic mitral valve are greater than, or “oversized” relative to, corresponding dimension(s) of the opening by a known amount or value. For example, each virtual implant model can have or be assigned a determined oversizing value, with this oversizing value being indicative of the oversizing conditions under which the model was generated. In some embodiments, the oversizing value can be a percentage and can be determined by Equation 1 above. By way of non-limiting example, a virtual implant model of a prosthetic mitral valve deployed within a mitral annulus-like opening under circumstances where the engagement region of the prosthetic mitral valve has a diameter, such as an anterior-posterior (AP) diameter, commissure-commissure (CC) diameter, equivalent diameter (i.e., (AP+CC)/2), that is known to be 20% greater than the corresponding diameter of the opening can be assigned an oversizing value of “20” or “20%”.

In some embodiments, each of the virtual implant models has two (or more) known geometric attributes, for example ellipticity and oversizing value. Other geometric attributes can alternatively be employed. The step 208 of selecting the virtual implant model of the candidate prosthetic mitral valve can thus include comparing the assessed ellipticity of the subject's native mitral annulus and an oversizing parameter of the candidate prosthetic mitral valve relative to the subject's native mitral annulus with ellipticities and oversizing values of the virtual implant models of the library otherwise corresponding with the candidate prosthetic mitral valve to identify or pick a most-appropriate virtual implant model for subsequent evaluations.

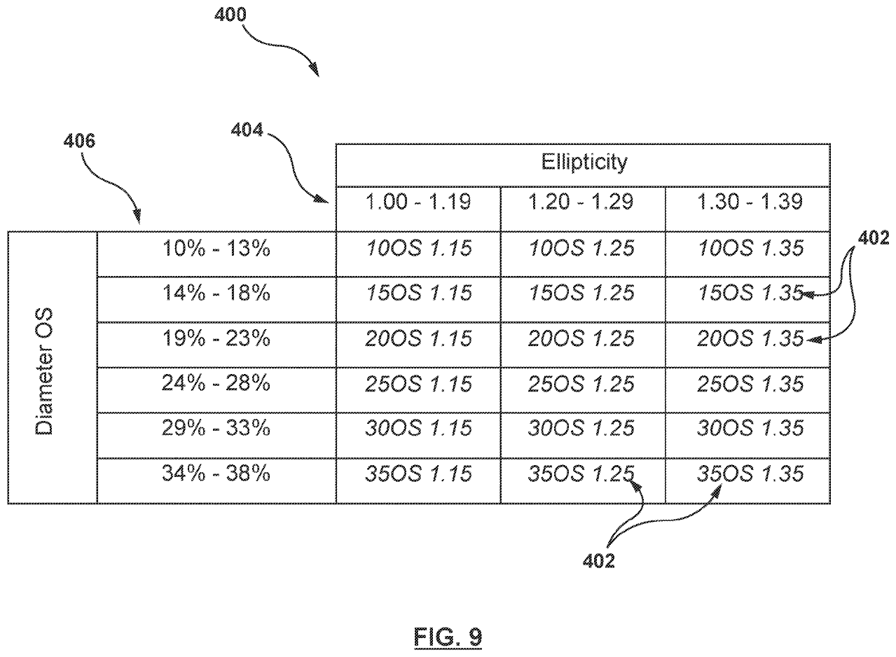

In some embodiments, the implant model library module 124 can be programmed to, or operate one or more protocols formatted to, facilitate selection of a most-appropriate virtual implant model from the library 150, for example via a lookup table that categorizes all of the virtual implant models in the library 150 for a particular available prosthetic mitral valve by the virtual implant models' known geometric attributes. Selection of the virtual implant model for subsequent evaluations can thus entail matching the assessed native mitral annulus of the subject and candidate prosthetic mitral valve with the known geometric attributes provided with the lookup table. One non-limiting example of a lookup table 400 in accordance with principles of the present disclosure is shown in FIG. 9. The lookup table 400 categorizes eighteen different virtual implant model designators (several of which are identified at 402) in terms of ellipticity and oversizing. In the example of FIG. 9, oversizing is in reference to equivalent diameter oversizing. As a point of reference, the name or title provided for each of the virtual implant model designators 402 in the table 400 identifies the known oversizing and ellipticity geometric attributes of a virtual implant model corresponding with the designator 402. For example, “25OS 1.15” identifies a virtual implant model representing the corresponding prosthetic mitral valve deployed within an opening having an ellipticity of 1.15 under conditions where the equivalent diameter of the prosthetic mitral valve is 25% larger than the equivalent diameter of the subject's native mitral annulus (i.e., an oversizing value of 25 or 25% from Equation 1).

The lookup table 400 assigns each of the virtual implant model designators 402 to one of a plurality of ranges of ellipticity 404, and to one of a plurality of ranges of oversizing values (“Diameter OS”) 406. The specific ellipticity ranges 404 and oversizing ranges 406 shown in FIG. 9 are merely exemplary and can be determined or defined in various fashions. In some embodiments, the ranges 404, 406 provided with the lookup table 400 can be established as a function of, or based upon, the available virtual implant models and distribution of the corresponding known geometric attributes. Regardless, by applying the subject's assessed native mitral valve ellipticity and assessed oversizing parameter to the lookup table 400, the most-appropriate virtual implant model is easily identified. For example, if the assessed equivalent diameter oversizing parameter is 23% and the assessed native mitral valve ellipticity is 1.02, the lookup table 400 identifies virtual implant model “200S 1.15” for selection.

Where provided, the lookup tables of the present disclosure are not limited to the format implicated by FIG. 9, and can alternatively utilizes geometric attributes other than one or both of ellipticity and equivalent diameter oversizing. With embodiments in which the implant model library module 124 (FIG. 5) incorporates or includes a lookup table or similar tool, a lookup table can be provided for each set of virtual implant models that otherwise correspond with a particular prosthetic mitral valve. In other embodiments, the lookup table can be universally applicable to two (or more) different prosthetic mitral valves, for example by presenting parameters that are independent of prosthetic mitral valve size and/or are generally applicable across two or more prosthetic mitral valve sizes. For example, the ellipticity parameters of the table 400 are independent of prosthetic mitral valve size. Further, the equivalent diameter oversizing parameters of the table 400 can be generally applied across differently-sized prosthetic mitral valves.

By way of further explanation, and continuing the PMV-A, PMV-B example above, the virtual implant model library 150 (FIG. 5) can have eighteen virtual implant models of PMV-A, with the virtual implant models corresponding with the geometric attributes associated with the virtual implant model designators 402 of FIG. 9. In other words, the set of virtual implant models for PMV-A include a first model that represents PMV-A deployed in an opening with an ellipticity of 1.15 and 10% oversizing conditions (i.e., “10OS 1.15” in the table 400), a second model that represents PMV-A deployed in an opening with an ellipticity of 1.15 and 15% oversizing conditions (i.e., “15OS 1.15” in the table 400), etc. The virtual implant model library 150 can further have eighteen virtual implant models of PMV-B that also correspond with the virtual implant model designators 402. For example, the set of virtual implant models for PMV-B include a first model that represents PMV-B deployed in an opening with an ellipticity of 1.15 and 10% oversizing conditions (i.e., “100S 1.15” in the table 400), a second model that represents PMV-B deployed in an opening with an ellipticity of 1.15 and 15% oversizing conditions (i.e., “15OS 1.15” in the table 400), etc. It will be understood that the virtual implant model of PMV-A deployed in an opening with an ellipticity of 1.15 and 10% oversizing conditions differs from the virtual implant model of PMV-B deployed in an opening with an ellipticity of 1.15 and 10% oversizing conditions. For example, where PMV-A and PMV-B are similarly-styled prosthetic mitral valves, but PMV-B has a larger outer diameter as compared to PMV-A, a footprint of PMV-A upon final deployment or implant will differ from a footprint of PMV-B upon final deployment. Regardless, under these and similar circumstances, the step 208 can entail applying the subject's assessed native mitral valve ellipticity and assessed oversizing parameter to the lookup table 400 as described above to determine or select the corresponding virtual implant designator 402. The virtual implant model from the set of implant models for the candidate prosthetic mitral valve that otherwise corresponds with the determined virtual implant designator 402 is selected for subsequent evaluations. For example, where the candidate prosthetic heart valve is PMV-B (as selected at the step 206), the subject's native mitral annulus is assessed to have an ellipticity of 1.27, and PMV-B was assessed to be 17% oversized relative to the subject's native mitral annulus, the table 400 implicates designator “15OS 1.25”; the virtual implant model of PMV-B corresponding with 150S 1.25 is then selected for subsequent evaluations.

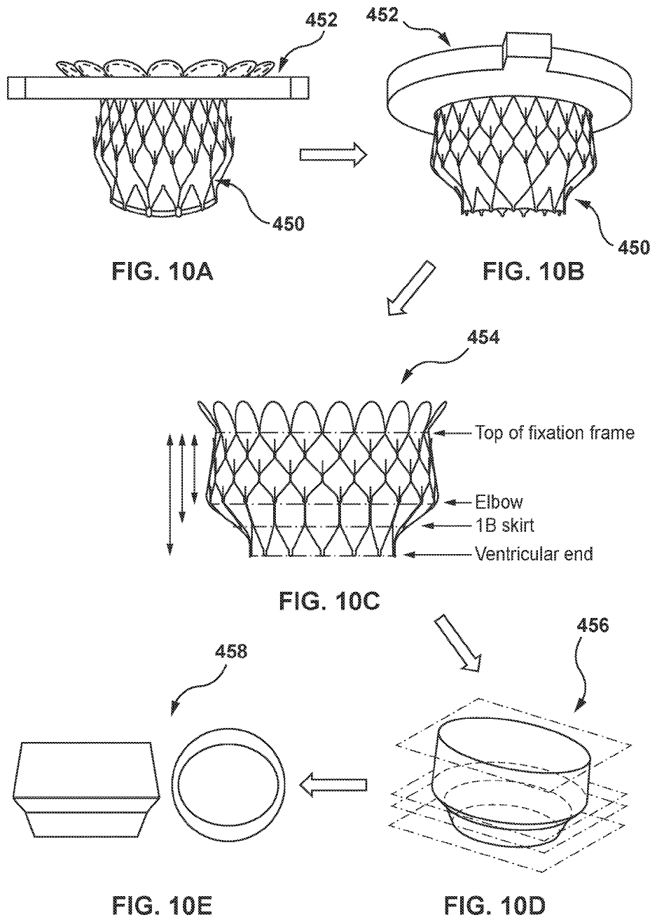

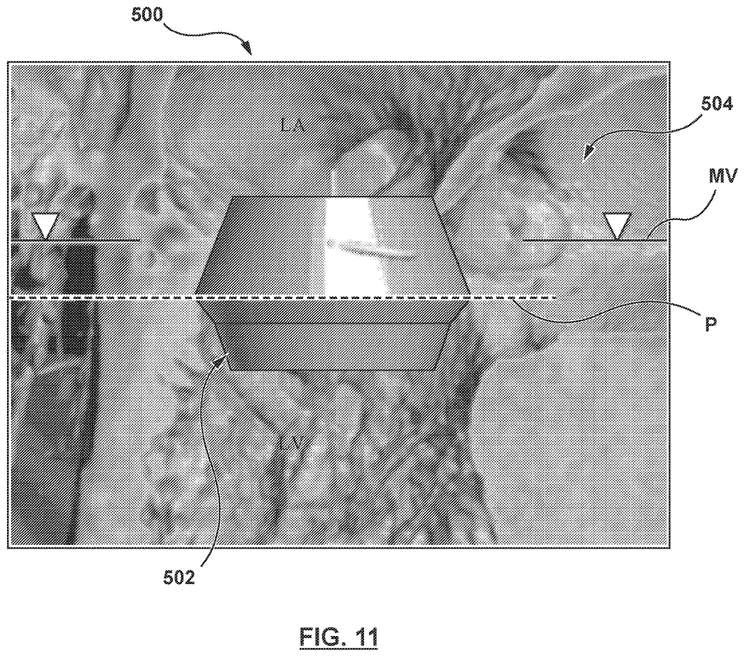

The virtual implant models of the library 150 can be created or generated in various fashions. In some non-limiting examples, the virtual implant models can be based upon modeling of an available prosthetic mitral valve physically deployed into a rigid fixture opening having mitral annulus-like shape with known dimensions and ellipticity. For example, FIG. 10A depicts an available transcatheter prosthetic mitral valve 450 (an Intrepid™ transcatheter mitral valve replacement (TMVR) device available from Medtronic, Inc.) deployed within an opening of a fixture 452. Dimensions and ellipticity of the opening of fixture 452 are predetermined and known, and can be selected based on various factors described below. In FIG. 10B, the prosthetic mitral valve 450 (and the fixture 452) has been placed in a warm water bath (e.g., 37 °C) to ensure complete expansion. Following complete expansion, images of the deployed prosthetic mitral valve 450 can be obtained, and a model generated from the image(s). For example, micro-CT scan(s) can be performed on the deployed prosthetic mitral valve 450, and CAD wireforms of the prosthetic mitral valve's wires can be constructed from the image(s) (e.g., STL file format). As shown in FIG. 10C, circumferential splines can be created at one or axial locations of the wireframe 454, for example at the top of the fixture, the elbow (or other geometric feature of the particular prosthetic mitral valve), the skirt edge (or other feature of interest of the particular prosthetic mitral valve), and the ventricular end. The so-created splines can then be projected onto a plane of best fit to obtain a 2D projected curve. Area, perimeter, major and minor diameters can be determined or calculated from the projected curves. Additional dimensional attributes, such as distance between the elbow, skirt edge and ventricular end plane to the top of the fixture can be determined or calculated (e.g., by measuring the distance between the spline centroids in a direction normal to the opening/annulus plane). From these (and perhaps other) dimensions, a solid 3D CAD model can be created, as shown at 456 in FIG. 10D. The solid 3D CAD model can be further refined by creating, on the front plane, an ellipse representing the top of the fixture, with the center of the ellipse at (0,0). Three additional planes can then be created parallel to and below the front plane to create ellipses representing the elbow, the skirt edge, and the ventricular end of the model. The ellipses can then be connected to create a final 3D model as shown at 458 in FIG. 10E.

A number of other techniques can be used to generate or create a 3D model of the prosthetic mitral valve 450 as deployed within the opening of the fixture 452. In yet other embodiments, a basis for the virtual implant models of the present disclosure can be something other than images of a prosthetic mitral valve physically deployed within a fixture. With optional embodiments utilizing the fixture-based techniques described above, a set of virtual implant images for a particular prosthetic mitral valve can be created by preparing a number of fixtures with varying opening dimensions and ellipticities; a virtual implant model of the prosthetic mitral valve as deployed at each of the differently-sized opening can be generated as described above. The variation in opening geometry from fixture-to-fixture can be selected to encompass likely end use applications. For example, fixture openings for a particular prosthetic mitral valve can be formatted to provide an equivalent diameter oversizing varying from 10%-35% in increments of 5%, and ellipticities of 1.15, 1.25, and 1.35. A wide variety of other fixture parameters can also be employed.

Returning to FIGS. 5 and 6, the virtual implant models of the library 150 can be generated in a number of other fashions that may or may not entail modeling of a prosthetic mitral valve physically deployed into a fixture. For example, the virtual implant models can be created virtually (e.g., finite element stimulation).

Generating the virtual implant representation at the step 210 can be performed in various manners, such as by the virtual implant module 126 by applying or overlaying the selected virtual implant model to the obtained image(s) of the subject's mitral valve and nearby anatomy. In this regard, the virtual implant model is arranged relative to the subject's anatomy so as to correspond with an expected location were the prosthetic mitral valve represented by the virtual implant model actually implanted within the subject. Thus, overlaying or applying the virtual implant model is performed as a function of geometrical features of the prosthetic mitral valve (and thus the virtual implant model of the prosthetic mitral valve) and the subject's anatomy. By way of reference, FIG. 11 provides an example virtual implant representation 500 of a selected virtual implant model 502 overlaid to an image 504 of a subject's cardiac anatomy. The example virtual implant model 502 includes or defines a landmark plane P that otherwise corresponds with an elbow-type geometric feature of the corresponding prosthetic mitral valve. As shown, the virtual implant model 502 has been located such that the elbow plane P is at a predetermined distance from a primary upper plane MV of the native mitral valve in the image 504 (e.g., depending upon a particular configuration of the particular prosthetic mitral valve upon which the virtual implant model 502 is based, the virtual implant model 502 can be arranged such that the elbow plane P is 8 mm, 9 mm, or some other predetermined dimension, from the mitral valve plane MV).

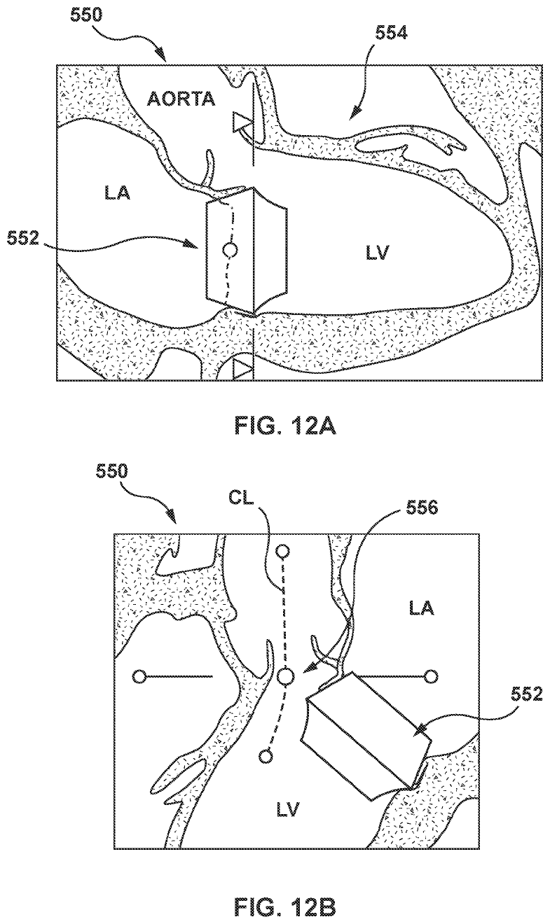

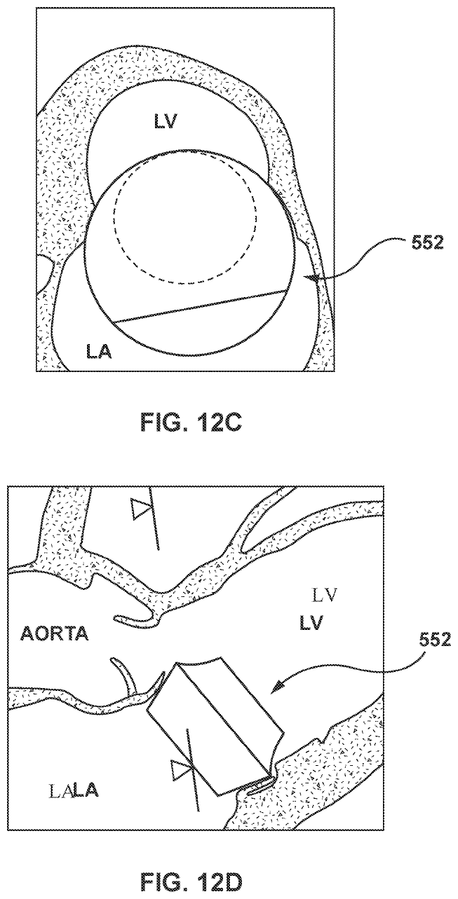

Returning to FIGS. 5 and 6, once the virtual implant model has been placed into the anatomy imaging, a neo-LVOT of the resultant virtual implant representation can be identified and reviewed. In particular, an area of the neo-LVOT is measured and reviewed at step 212 in various manners. For example, FIG. 12A depicts virtual implant representation 550 in which a virtual implant model 552 has been overlaid or applied to an obtained image 554 of a subject's anatomy. In some embodiments, and with reference to FIG. 12B, an aortic centerline CL is defined in the virtual implant representation 550 that passes through the new LVOT 556 (referenced generally) created by placement of the virtual implant model 502. The smallest neo-LVOT area can be visually identified along the aortic centerline CL by sweeping through the space between the virtual implant model 502 and the ventricular from the inflow to the outflow. The smallest neo-LVOT area can be measured at cross-sectional images corresponding to the minimum area location (e.g., via a CAD polygon tool), examples of which are provided in FIGS. 12C and 12D.

Returning to FIGS. 5 and 6, other techniques can be employed to review the neo-LVOT, and parameters other than smallest neo-LVOT area can be determined and/or considered by, for example, the measurement and analysis module 128. Similarly, the evaluation or determination at 214 as to whether or not the candidate prosthetic heart valve is appropriate for the subject can be based on various factors. In some embodiments, the determined smallest neo-LVOT area can be compared to a predetermined threshold or cut-off criteria. If the determined neo-LVOT area less than the cut-off criteria or value, it can be determined that the candidate prosthetic heart valve (again, that otherwise forms the basis for the selected virtual implant model) is not appropriate for the subject. The present disclosure is not limited to any one particular cut-off criteria. In some examples, the cut-off criteria can be a neo-LVOT area less than 1.5 cm² at end-systole, 1.6 cm² at early-systole, etc. From these explanations, the virtual implant representation generation and review as described above can be formatted or selected to coincide with any contextual parameters of the cut-off criteria. For example, where the cut-off criteria is a minimum neo-LVOT area at end-systole, then the virtual implant representation generated at the step 210 and reviewed at step 212 can include the selected virtual implant model being overlaid or applied to images of the subject's native mitral valve anatomy at an end-systole stage of the cardiac cycle. Under circumstances where two (or more) cut-off criteria are available for consideration, with each criteria implicating a different cardiac cycle stage, systems and methods of the present disclosure can include generating and evaluating virtual implant representations, as described above, of the selected virtual implant image as applied to image(s) of the subject's anatomy at each of the cardiac cycle stages of the criteria.

It should be understood that various aspects disclosed herein may be combined in different combinations than the combination specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purpose of clarity, it should be understood that the techniques of this disclosure may be performed by a combination of units or modules associated with, for example, a medical device.

In one or more examples, the described techniques may be implemented in hardware, software, firmware, or a combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

The systems and methods of the present disclosure provide a marked improvement over previous designs. By utilizing a virtual implant model that closely resembles a particular prosthetic mitral valve actually deployed to a subject's native mitral valve, reliable evaluations of an appropriateness of the particular prosthetic mitral valve for the particular patient can be made. For example, a reliable evaluation of a likely neo-LVOT created by a particular prosthetic mitral valve relative to the subject's native anatomy can be achieved. Further, the systems and methods of the present disclosure may decrease the overall CT (or other) screening time of the subject (as compared to conventional techniques) by, for example, reducing the need for multiphasic LVOT analysis.

Although the present disclosure has been described with reference to preferred embodiments, persons skilled in the art will recognize that changes can be made in form and detail without departing from the spirit and scope of the present disclosure. For example, while some embodiments described above implicate a prosthetic mitral valve and neo-LVOT evaluations, the systems and methods of the present disclosure are equally applicable to prosthetic tricuspid valves and neo-RVOT evaluations.

What is claimed is:

1. A method for evaluating a human subject having indications for receiving a prosthetic valve, the method comprising:
obtaining images of a native valve and surrounding anatomy of the subject;
assessing a native annulus of the native valve via the obtained images;
selecting a candidate prosthetic valve from a plurality of available prosthetic valves based upon the native annulus assessment;
selecting an implant model of the candidate prosthetic valve from a library of different implant models based upon the native annulus assessment;
generating a virtual implant representation by applying the selected implant model to the obtained images, the virtual implant representation having a neo-VOT (ventricular outflow tract);
reviewing an area of the neo-VOT; and
evaluating whether the candidate prosthetic valve is appropriate for the subject based upon the review.

2. The method of claim 1, wherein the prosthetic valve is a prosthetic mitral valve, the native valve is a native mitral valve, the candidate prosthetic valve is a candidate prosthetic mitral valve, the plurality of available prosthetic valves are a plurality of available prosthetic mitral valves, and the neo-VOT is a neo-LVOT (left ventricular outflow tract).

3. The method of claim 2, wherein the step of assessing includes determining at least one parameter of the native annulus selected from the group consisting of annulus perimeter, anterior-posterior diameter, commissure-commissure diameter, and annulus area.

4. The method of claim 3, wherein the step of assessing includes performing the step of determining at a plurality of phases of a cardiac cycle of the subject.

5. The method of claim 2, wherein each of the prosthetic mitral valves of the plurality of available prosthetic mitral valves defines an annulus engagement region configured to engage an annulus of a patient's mitral valve upon implantation, and further wherein step of selecting a candidate prosthetic mitral valve includes identifying a prosthetic mitral valve from the plurality of available prosthetic mitral valves includes comparing a size of a determined perimeter of the native annulus with a size of the annulus engagement region of at least some of the prosthetic mitral valves of the plurality of available mitral valves.

6. The method of claim 5, wherein the step of selecting a candidate prosthetic mitral valve includes identifying a prosthetic mitral valve from the plurality of available prosthetic mitral valves having the corresponding annulus engagement region size that is greater than the size of the determined perimeter of the native annulus.

7. The method of claim 6, wherein the step of selecting a candidate prosthetic mitral valve includes identifying a prosthetic mitral valve from the plurality of available prosthetic mitral valves having the corresponding annulus engagement region size that is 10-30% greater than the size of the determined perimeter of the native annulus.

8. The method of claim 2, wherein the implant models of the library include a plurality of implant models for the candidate prosthetic mitral valve each having an assigned oversizing value, and further wherein the step of selecting an implant model includes comparing an oversizing parameter of the selected candidate prosthetic mitral valve relative to the native annulus with the assigned oversizing values.

9. The method of claim 8, wherein the oversizing parameter is based upon an anterior-posterior diameter and commissure-commissure diameter of the native annulus.

10. The method of claim 8, wherein the step of selecting an implant model includes identifying the assigned oversizing value closest to the oversizing parameter.

11. The method of claim 8, wherein each of the implant models of the plurality of implant models for the candidate prosthetic mitral valve has an assigned ellipticity value, and further wherein the step of selecting an implant model includes comparing an ellipticity parameter of the native annulus with the assigned ellipticity values.

12. The method of claim 11, wherein the step of selecting an implant model includes identifying the assigned ellipticity value closest to the ellipticity parameter.

13. The method of claim 1, wherein the library includes a first virtual representation of a first available prosthetic mitral valve deployed in an opening of a first fixture and having a first opening diameter and a first ellipticity, and a second virtual representation of the first available prosthetic mitral valve deployed in an opening of a second fixture having a second opening diameter and a second ellipticity.

14. The method of claim 2, wherein the step of generating includes arranging a known feature of the selected implant model relative to an anatomical landmark of the native mitral valve.

15. The method of claim 14, wherein the anatomical landmark is a plane of the native annulus.

16. The method of claim 15, wherein the known feature is an elbow formed the available prosthetic mitral valve at an outflow side thereof.

17. The method of claim 2, wherein the step of reviewing includes:
identifying an aortic centerline in the neo-LVOT;
identifying a minimum neo-LVOT region along the aortic centerline in a plane perpendicular to the aortic centerline; and
measuring an area of the minimum neo-LVOT region.

18. The method of claim 17, wherein the step of evaluating includes determining whether the measure area is less than a predetermined value.
